(19) United States
(12) Patent Application Publication
(10) Pub. No.: US 2004/0120894 A1
(43) Pub. Date: Jun. 24, 2004

(54) Rodenticide and method of screening

(76) Inventor: William Dawson, Alton (GB)

Correspondence Address: LADAS & PARRY, 26 WEST 61ST STREET, NEW YORK, NY, 10023, US

(21) Appl. No.: 10/250,796

(22) PCT Filed: Jan. 16, 2002
(86) PCT No.: PCT/GB02/00171

(51) Int. Cl. A61K 49/00; A01N 25/00

(52) U.S. Cl. 424/9.2; 424/405

(57) ABSTRACT

A rodenticide which is non-toxic to humans, domestic animals and livestock comprises a water-retentive material as the active ingredient and a rodent attractant. Preferably the water-retentive material is cellulosic material. In certain embodiments the water-retentive material comprises alpha-cellulose. In one preferred embodiment the cellulosic material comprises purified cellulose derived from the core of the cob of the DK 446 maize hybrid or from the core of the cob of an agonist of the DK 446 hybrid. It is considered that the unique selective toxicity of such rodenticides arises from the interference with water transport through the gut wall, particularly in the caecum (where cellulose is digested in rats) The caecum is vestigial in humans, who are not therefore affected by such materials. A laboratory method of screening candidate water-retentive materials for rodenticidal activity in the field is disclosed, involving examination of the gut (for compaction and bloating) and the caecum (for impaction).

The present invention relates to rodenticides and to methods of screening for rodenticidal activity.

In our patent GB 2,311,464B a novel class of rodenticides is disclosed, based on cellulosic material derived from (eg the core of the cobs of) certain hybrids of maize, namely the Dekalb DK 446 hybrid and agonists thereof. The cellulosic material constitutes the active rodenticidal material and the disclosed rodenticides are non-toxic to humans, domestic animals and livestock.

Although some symptoms of the test animals are given in the above patent specification, and excretion of fat and adipose tissure was presented as a particularly important symptom, the precise mode of action of the above rodenticides has remained unclear. Yet other hybrids besides the Dekalb DK 446 hybrid have been found by the present applicants to be effective as rodenticides in the field, and, like the rodenticide based on the DK 446 hybrid, are non-toxic to humans and livestock. Surprisingly, some hybrids which are effective as rodenticides in the field are not effective in killing rats under laboratory conditions.

An object of the present invention is to provide a method of screening candidate materials (particularly carbohydrate materials and especially maize hybrids) for rodenticidal activity.

It has now been found that the above rodencticides retain water in the gut and that the resulting disruption of water transport through the gut wall in turn interferes with the normal digestion of the rodent. Among the various methods of water transport into and out of cells, passage through ion channels is one of the important processes. These channels vary amongst mammals—ie there is a species variation such that mice are most sensitive and humans least to changes in extracellular water environment with rats intermediate. It has now been found that cellulosic material is particularly effective at modifiying ion transport in rodents. Consequently the rodent is weakened and dies.

Furthermore in rats, digestion of cellulose takes place mainly in the caecum as a result of the action of bacteria in that region of the gut, whereas in humans the caecum is vestigial and has no digestive action on cellulose. It is now considered that water-retentive cellulosic materials, when ingested by rats, compromise their digestion in the caecum. Such materials have no effect on humans or livestock.

It has been unexpectedly found that the thymus is reduced in size in rats which have been fed the above cellulosic rodenticides, indicative of a compromised immune system, and it is envisaged that other water-retentive carbohydrate materials, particularly cellulose will have a similar effect. It is considered that their water-retentive effect disrupts ion transport through ion channels in the wall of the gut.

Accordingly, in one aspect the invention provides a rodenticide comprising a water-retentive material as the active ingredient and a rodent attractant.

Preferably the water-retentive material is cellulosic material.

In certain embodiments the water-retentive material comprises alpha-cellulose. In one preferred embodiment the cellulosic material comprises purified cellulose derived from the core of the cob of the DK 446 maize hybrid or from the core of the cob of a hybrid related to the of an agonist of the DK 446 hybrid.

The invention also provides method of making a rodenticide comprising the steps of combining a water-retentive material with a rodent attractant, the water-retentive material being the active ingredient of the rodenticide.

Cellulose is the most water-retentive of the complex carbohydrates—it is more water-retentive than starch for example. Moreover cellulose in particular retains its three-dimensional structure in the gut and can thereby provide centres for microbial proliferation. In practical terms, it has now been found that such materials reduce the number of immuno-competent cells when ingested by rodents. In particular the generation of T-lymphocytes in the thymus of rodents is inhibited by ingestion of water-retentive materials.

Any of these effects can be used as a basis for a method of screening candidate rodenticides in accordance with an aspect of the invention.

As noted above, in rats, digestion of cellulose takes place mainly in the caecum as a result of the action of bacteria in that region of the gut, whereas in humans the caecum is vestigial and has no digestive action on cellulose. It is now considered that water-retentive materials, when ingested by rats, compromise their digestion in the caecum and weaken their immune system. However under laboratory conditions, a weakened, immuno-compomised rat will be much more likely to survive than in the wild, since in the laboratory cage food and water will be readily available, there will be no predators and there will be a reduced possiblity of infection.

Typically, we have found that under laboratory conditions, ie free of predators and confined, rats fed ad libitum with cellulosic materials which are effective in the field as rodenticides lose weight in the initial phase of testing and then recover and survive the test.

More particularly, our studies indicate that a water-retentive candidate material (preferably cellulosic material, especially a maize hybrid) may be effective in the field if, under laboratory conditions, rodents (preferably laboratory rats or laboratory mice, but less preferably wild rats or wild mice) fed ad libitum with the candidate material (particularly cellulosic material obtained from a maize hybrid) exhibit a mean weight loss of at least 15% (preferably at least 20%, more preferably at least 25%, most preferably at least 30%) of initial body weight during the initial phase (eg the first five days) of testing.

Accordingly, in another aspect, the invention provides a method of screening water-retentive candidate materials for potential rodenticidal activity in the field, the method comprising providing water-retentive materials as candidate materials, feeding rodents with the candidate materials ad libitum under laboratory conditions, measuring weight loss in the rodents during an initial phase of the testing, and selecting those:candidate materials which lead to a mean weight loss of at least 15% (preferably at least 20%, more preferably at least 25%, most preferably at least 30%) of initial body weight.

In a related aspect, the invention provides a method of screening water-retentive materials for rodenticidal activity, wherein a water-retentive material is fed to rodents and the rodents are tested to determine whether or to what extent the water-retentive material has disrupted water transport through the wall of the gut.

Preferably the rodents are tested to determine whether or to what extent the water-retentive material has disrupted ion transport through the wall of the gut.

Preferably the effect of ingesting the water-retentive material on the size or condition of the thymus gland is tested.

Preferably said water-retentive material is of natural origin.

Preferably said water-retentive material is cellulosic material.

Preferably the water-retentive material is derived from corn-cobs.

Preferably the rodents are examined post mortem.

In view of the newly discovered mode of action, the invention is not limited to water-retentive materials derived from corn-cobs.

Accordingly, in another aspect the invention provides a rodenticide comprising cellulosic water-retentive material as the active ingredient and a rodent attractant, the cellulosic water-retentive material being substantially free of corn-cob material.

In this aspect the invention also provides a rodenticide comprising cellulosic water-retentive material as the active ingredient and a rodent attractant, the cellulosic water-retentive material being substantially free of material derived from the core of the cob of the DK 446 maize hybrid or from the core of the cob of an agonist of the DK 446 hybrid.

Also in this aspect the invention provides a method of making a rodenticide, the method comprising the step of combining cellulosic water-retentive material with a rodent attractant, the cellulosic water-retentive material being substantially free of corn-cob material and being the active ingredient of the rodenticide. Preferably the cellulosic water-retentive material is substantially free of material derived from the core of the cob of the DK 446 maize hybrid or from the core of the cob of an agonist of the DK 446 hybrid.

Preferably at least the water-retentive material is dried, preferably under conditions of elevated termperature and/or pressure.

The rodenticides of the present invention are preferably combined with a sweet material such as molasses (which acts as a rodent attractant) and pelletised by the methods disclosed in GB 2,311,464, which is incorporated herein by reference. The pellets are preferably packaged in moisture-proof bags in order to preserve the dry state (and hence water-absorbing) properties of the rodenticide.

It is envisaged that a wide variety of water-retentive materials may be suitable for use as rodenticides. The most useful materials are expected to be non-toxic (to humans) materials of natural origin such as celluloses whose water-retentive properties arise from their macromolecular structure. Other suitable materials can be found by experiment.

Accordingly, in another aspect the invention provides a method of screening water-retentive materials for rodenticidal activity, wherein a water-retentive material is fed to rodents (preferably laboratory rats) and the rodents are tested to determine whether or to what extent the water-retentive material has disrupted water transport through the wall of the gut.

Such disruption of water transport can be detected by its effects, eg inhibition of digestion, which can be determined by examination of the gut.

Preferably the rodents are examined postmortem.

Preferably the rodents are examined for intestinal bloating.

Optionally the effect of ingesting the water-retentive material on the size or condition of the thymus gland is tested.

Preferably the water-retentive material is substantially non-toxic to humans.

Other preferred features are defined in the dependent claims.

In another aspect the invention provides a method of alleviating rodent infestation comprising depositing in an area of rodent infestation a rodenticide as claimed in claim 16, the rodenticide being non-toxic to humans and disrupting the digestion of the rodents on being ingested.

Methods of testing in accordance with the invention are described below with reference to the following examples which detail separate studies performed by independent testing laboratories.

### EXAMPLE 1

Purpose

The purpose of this study was to determine the effectiveness of ERADIMOUSE for control of Norway rats (Rattus norvegicus). This was 15-day no-choice test, with a 5-day post-test observation period, to determine the laboratory efficacy.

Test Substance

The test substance was ERADIMOUSE, a pelleted rodenticide (5 mm (3/16″) diameter pellets) which is a commercial product of the applicants that is non-toxic to humans or livestock and which has proven effective in the field. It consists of 95 wt % white core corn cob material derived from “Corn Cobb 20-40 grind” obtained commercially from Mount Pulaski Products Inc., 908 N. Vine St., Mount Pulaski, Ill. 62548, USA and 5 wt % black strap molasses and is made in accordance with the process described in our GB 2,311,464B patent.

Test System

Species—Norway Rat (Rattus norvegicus).

Strain—Wild type, originally from the Chicago, Ill. area, trapped during spring and summer of 2000, and placed in outdoor colony.

Source of Supply—The rats were live trapped using appropriate size live traps (Tomahawk® or similar) at a captive colony in Colorado. The rats were placed in holding cages. All rats in holding were monitored daily to assure feed and water were available ad libitum, and bedding changes were performed twice each week.

Justification for Selection—The Norway rat (wild type) was used because the source population was identified as having a high proportion of rats with genetic resistance to warfarin rodenticides.

Size—The mean rat weight on Day 0 was 297.1 g (+13.4 SE). Among the test groups, the females averaged 246.2 g (+20.0 SE) and males averaged 331.8 g (+16.9 g), whereas the control males were 313.4 g (+24.2 SE).

Age—Any ages of rats was acceptable, as long as the minimum weight criterion was met.

Sex and Number—The treatment group consisted of 10 males and 10 females. The control group was all males (n=10).

Physical Condition—The test animals were healthy and active.

Housing, Identification and Environmental Conditions of the Test System

Housing and Environment—A test room was reserved exclusively for the rats used in this study. Rats were maintained in screen bottom metal cages having a surface area of approximately 972 cm² during the test. All rats were kept in individual cages for the duration of this study. Cages were labeled so that the cage number corresponds with the test animal number on the data collection forms.

A photoperiod of 12 hours light: 12 hours dark was maintained for the duration of the test. The room was lighted with red incandescent bulbs, since research has shown that red light lessens stress of captive wild rats (Fall, 1974). The high and low temperature and humidity readings were recorded daily using a Fisher Scientific Thermo-Hygro measuring device.

Feed and Water of the Test System

Acclimation—Fresh Teklad F6 Rodent Diet (W) 8664 (Harlan Teklad, Madison, Wis.) was available ad libitum to all rats during acclimation and the post-test period. Water from bottles equipped with stainless steel sipper tubes was available ad libitum throughout the acclimation, test phase, and post-test period.

Test—The EradiMouse test substance was provided to the treatment group rats during the 15- day test period. Control group rats were offered the Teklad F6 Rodenti Diet. Water was available ad libitum.

Post-test—The Fresh Teklad F6 Rodent Diet was provided at all times during the 5-day post-test observation period or any extended period. Water from bottles equipped with stainless steel watering tubes was provided ad libitum.

Experimental Design

Number of Animals Per Group—The test group consisted of 20 Norway rats (10 males and 10 females). An additional group of 10 male rats served as the control group.

Group Assignment—The male rats were assigned to treatment and control groups using a computerized randomization program (Ran30).

Feed Consumption—Feed consumption was monitored during the 5-day acclimation period, 15-day exposure period, and 5-day post-treatment recovery period.

Body Weight—Body weights were recorded during acclimation, when dead animals were found, and when rats were euthanized.

Necropsy, Microbiological Analysis, and Histology—Animals from the treatment group that died during study were subjected to gross necropsy, on the day of death, for signs of toxicosis or other cause of death. Likewise, wheneyer a male rat died a control rat was randomly selected and euthanized. The controls were also necropsied. Organ tissues including the gastrointestinal tract (from esophagus to colon, with the ends tied off to prevent spillage), liver, and kidneys were extracted and placed in labeled plastic bags, and the organ samples were taken to the Colorado State University Veterinary Hospital Diagnostic Laboratory. Contents from the GI tract were analyzed for Clostridium species, as well as for other anaerobic and aerobic microbes. Histological tissues from the GI tract, liver, and kidney were analyzed to evaluate for any lesions or abnormalities.

Observations—All rats were observed daily during acclimation, exposure, and the post-test period for signs of toxicosis, morbidity, or mortality.

Statistical Methods

Data on the environment, observations, mortality and necropsy were tabulated and presented descriptively. Daily food consumption data for acclimation and test period were analyzed separately, using a 2-way repeated measures ANOVA, with food consumption as the dependent variable, diet group (male treatment group, female treatment group, and male control) as the test factor, and time as the repeated factor.

Results and Discussion

Temperatures within the test room were maintained between 20 and 29° C., and humidity ranged from 29 to 72% during the study period.

Food Consumption—During acclimation, the male (15.4 g/day+0.8 SE) and female 17.3 g/day+1.2 SE)) rats from the treatment groups consumed slightly less feed than the male rats from the control group (20.7 g/day+1.4 SE) (F2, 27=5.21, P=0.01). Post hoc comparison (Tukey's test) indicated that the male treatment group consumed less food per day than the control (P<0.05), but the there was no difference between females and either the control group those that were scheduled for treatment. Note that the one of the males from the treatment group (rat #15) consumed <10 g of food during the first four days of acclimation, and this lack of consumption skewed the data. This rat consumed 26 g on days 5 and 6 of acclimation, thus we chose to include this animal with the treatment group.

Evaluation of food consumption during the definitive phase indicated a significant effect from the treatment (F2,405=3.11, P=0.046), time (F14,405=4.68, P<0.001), and the interaction of treatment and time (F28,405=3.19, P<0.001). The both males and females from the Eradimouse test groups appeared to consume less than controls during the first four days after beginning the test. Food consumption among groups was relatively equivalent during days 5-10, but the Eradimouse rats appeared to eat more than controls during days 11-15 of the test (FIG. 2). Post hoc analysis with Tukey's test indicate that the females consumed less than the controls on Days 1, 2, and 4 (P<0.05); whereas males consumed less than controls on Day 1 (P<0.05). The males and females consumed approximately the same amount of food each day (P>0.05), and the patterns of food consumption for each sex were similar FIG. 2). These data appear to suggest that the rats initially rejected the palatability of the

Eradimouse bait, but because of hunger they compensated for the loss by the end of the 15-day trial.

Body Weight—Of the animals that died during the test period (2 females and 5 males), the females lost an average of 35.5% of pre-test body weight, and the males lost 33.9% (combined=34.4%+4.3 SE). The 6 Eradimouse rats that were euthanized on Day 15 also lost substantial weight (22.1%+2.4 SE), but the weight loss of the euthanized was not as much as those that died (t=2.39, 11 dt P=0.0356). The 5 control males that were sacrificed during the test lost 1.9% of pretest body weight, and the additional 3 that were euthanized on Day gained 0.7%. Summary data are in Table 2.

Observations—The rats remained active through most of the trial. According to observation records one of the treated rats (#28) appeared moribund on day 14, and this animal was found dead 5.75 hours after initial observations. Other than appearing to lose weight, the rats did-not display any other clinical signs of illness.

Mortality—A total of 7 rats (35%) that were fed the Eradimouse bait died during the test and recovery period (5 males and 2 females). All 5 of the male rats died during the actual test phase. One of the females (#7) died on the first day of recovery the other died on Day 8 of the test.

Necropsy—Most of the rats did not exhibit any external abnormalities, although rat #15 had some lesions on its leg and chest and rat #20 had a small wound on its foot (Table 2). Of the 7 rats from the Eradimouse test group that died during the study, 6 exhibited empty but bloated gastrointestinal tracts. One of the rats (#26) had liver parasites. Three of the ten control rats that were sacrificed also had bloated or gas-filled gastrointestinal tracts, but these were full of food. Two control rats showed liver abnormalities, including one with yellow spots and the other with parasites. At the termination of the feeding test (Day 15), n=9 rats were euthanized (6 from the Eradimouse treatment group and 3 controls), necropsied, and tissue samples were sent to the Veterinary Diagnostic Laboratory. The three controls showed no internal abnormalities. Three of the six from the Eradimouse group had discoloration of the liver, and 3 of 6 had bloated gastrointestinal tracts. One rat exhibited no internal abnormalities.

References

Fall, M. W. 1974. The use of red light for handling wild rats. Laboratory Animal Science. 24:4.

TABLE 1
Body Weights (BW) of Norway rats during 15 day no choice test
with Eradimouse bait.

| ID | Sex | Diet | Died/Sac | BW before (g) | BW after (g) |
|---|---|---|---|---|---|
| 1* | F | E | Sac | 209.4 | 56.0 |
| 2 | F | E | Sac | 323.3 | 319.9 |
| 3 | F | E | Sac | 128.8 | 135.7 |
| 4 | F | E | Sac | 220.6 | 229.6 |
| 5 | F | E | Sac | 177.6 | 187.5 |
| 6 | F | E | Sac | 246.3 | 250.8 |
| 7 | F | E | Died | 260.0 | 159.1 |
| 8* | F | E | Sac | 287.9 | 226.4 |
| 9* | F | E | Sac | 282.1 | 225.4 |
| 26 | F | E | Died | 325.5 | 221.0 |
| 10* | M | E | Sac | 365.3 | 267.0 |
| 11* | M | E | Sac | 264.9 | 228.9 |
| 13 | M | E | Sac | 369.4 | 346.5 |
| 14 | M | E | Sac | 312.2 | 294.8 |
| 15 | M | E | Died | 336.1 | 302.3 |
| 17 | M | E | Died | 279.5 | 160.7 |
| 19 | M | E | Died | 299.6 | 189.3 |
| 22* | M | E | Sac | 362.1 | 251.8 |
| 23 | M | E | Died | 440.6 | 270.2 |
| 28 | M | E | Died | 288.5 | 168.4 |
| 12* | M | C | Sac | 320.5 | 320.5 |
| 16 | M | C | Sac | 253.1 | 270.5 |
| 18 | M | C | Sac | 285.7 | 314.1 |
| 20* | M | C | Sac | 355.8 | 352.3 |
| 21 | M | C | Sac | 394.6 | 292.4 |
| 24* | M | C | Sac | 299.6 | 309.0 |
| 25 | M | C | Sac | 435.7 | 416.1 |
| 27 | M | C | Sac | 178.1 | 198.1 |
| 29 | M | C | Sac | 249.8 | 232.5 |
| 30 | M | C | Sac | 361.4 | 332.4 |

*Rats were sacrificed on Day 15.

TABLE 2
Results of gross necropsy performed on Norway rats during test of Eradimouse bait.
Necropsy

| ID | Sex | Treatment | Died/Sac | External | Interna1 |
|---|---|---|---|---|---|
| 7 | F | Eradimouse | Died | No abnormalities | GI^{a} was very bloated; no food |
| 15 | M | Eradimouse | Died | Abrasions-chest & legs | Intestinal tract swollen; no food |
| 17 | M | Eradimouse | Died | No abnormalities | Stomach & Intestines bloated; no food |
| 19 | M | Eradimouse | Died | ″ | GI empty/bloated |
| 23 | M | Eradimouse | Died | ″ | GI bloated, empty |
| 26 | F | Eradimouse | Died | ″ | Liver parasites |
| 28 | M | Eradimouse | Died | ″ | GI bloated-LI black-no food in GI |
| 12 | M | Control | Sac | ″ | No abnormalities |
| 16 | M | Control | Sac | ″ | Some yellow spots observed on liver |
| 18 | M | Control | Sac | ″ | GI bloated; full |
| 20 | M | Control | Sac | Small wound on foot | No abnormalities |
| 21 | M | Control | Sac | No abnormalities | ″ |
| 24 | M | Control | Sac | ″ | ″ |
| 25 | M | Control | Sac | ″ | ″ |
| 27 | M | Control | Sac | ″ | LI^{b} was gassy (<treated), but full |
| 29 | M | Control | Sac | ″ | ″ |
| 30 | M | Control | Sac | ″ | Liver parasites |
| 1 | F | Eradimouse | Sac | ″ | Liver Discoloured (yellow) |
| 2 | F | Eradimouse | Sac | ″ | LI was distended, but full |
| 3 | F | Eradimouse | Sac | ″ | GI was bloated/gassy, but full |
| 4 | F | Eradimouse | Sac | ″ | GI was bloated/gassy, but full |
| 5 | F | Eradimouse | Sac | ″ | LI was distended, but full |
| 6 | F | Eradimouse | Sac | ″ | LI was distended/gassy, but full |
| 8 | F | Eradimouse | Sac | ″ | Liver Discol (yellow) |
| 9 | F | Eradimouse | Sac | ″ | No abnormalities |
| 10 | M | Eradimouse | Sac | ″ | LI-Large volume of diet |
| 11 | M | Eradimouse | Sac | ″ | LI-some bloating observed (<treated) |
| 13 | M | Eradimouse | Sac | ″ | LI was distended/gassy, but full |
| 14 | M | Eradimouse | Sac | ″ | LI/SI were both distended/gassy, full |
| 22 | M | Eradimouse | Sac | ″ | GI + stomach bloated, full; liver yellow spots |

^{a}GI = gastro-intestinal tract
^{b}LI = large intestine

Conclusion

The study shows that ERADIMOUSE interferes with the digestion of wild type rats and leads to weight loss but not necessarily death under laboratory conditions. Ingestion of the product causes intestinal bloating as a result of inhibition of normal digestion, in turn considered to be caused by disruption of water transport through the wall of the gut.

### EXAMPLE 2

Objective

To determine the effectiveness of the test substance to produce death in the treated rats when administered as supplied, ad libitum, for a period of 14 days.

Methods and Materials

1. Test Substance:

Identification: ERADIRAT, lot #020901.

This sample was pelleted feed in a white bag.

ERADIRAT a pelleted rodenticide (9 mm (⅜″) diameter pellets) which is a commercial product of the applicants that is non-toxic to humans or livestock and which has proven effective in the field. It consists of 94 wt % white core corn cob material obtained commercially from “Corn Cobb 20-40 grind” obtained commercially from Mount Pulaski Products Inc., 908 N. Vine St., Mount Pulaski, Ill. 62548, USA 5 wt % black strap molasses and 1 wt % wheatflour.

2. Test Animal:

Species: Rat (rattus norvegicus)

Strain: Sprague Dawley

Supplier:Harlan Sprague Dawley Indianapolis, Ind.

Number/Sex: 10 males

10 females (nulliparous and non-pregnant)

10 controls (5 males and 5 female)

During the acclimation and testing period, each animal was housed and maintained according to the “Guide for the Care and Use of Laboratory Animals” (NRC, 1996). All animals were identified by the ear hole-notch method and had cage cards which provided the individual animal and project numbers. The animals were singly housed in suspended wire cages and fed certified Purina Rodent Chow and county water, ad libitum. Note: The first day the rats were fed non-certified rodent chow. All animals were acclimated for 7 days prior to testing. Animals were observed for general health and suitability for testing during this period. Beginning the day after arrival, both feed and water consumption analyses were performed.

3. Experimental Procedure:

After the acclimation period, 20 Sprague Dawley rats (10 males, 10 females) were selected as the test system and 10 Sprague Dawley rats (5 males and 5 females) were selected as controls. Rats for each sex's test system were selected by calculating the mean body weight and determining the acceptable weight limit range (+/−20% of the mean weight). The initial body weights of the test animals ranged from 130.7 g to 148.2 g in the test females, 153.0 g to 169.6 g in the test males, 134.0 g to 145.2 g in the control females and 155.5 g to 162.5 g in the control males.

The test substance was administered orally ad libitum. The control animals received certified Purina Rodent Chow ad libitum.

Feed and water consumption were calculated daily. Each day the amount of food remaining in the hopper was weighed and recorded. If necessary more food was added to ensure ad libitum feeding and this total was recorded. Additionally, the food which dropped onto the absorbent pad under each cage was removed weighed and recorded for each animal. The amount consumed for each animal was calculated from these values.

Previous day's total food amount returned to hopper (g)−weight of food remaining in hopper (g)−weight of food recovered from absorbent pad (g)=Amount of Food Consumed (g)

Each day the water bottles were weighed and recorded. If necessary, more water was added to ensure ad libitum watering. The total amount was weighed and recorded. Amount consumed was calculated from these values.

Previous day's total water amount in bottle (g)−weight of water remain inv, in bottle for current day (b)=Amount of Water Consumed (g)

Note: 1.0 g is equivalent to 1.0 ml of water.

Averages for the acclimation and test period for both food and water consumption can be seen in Tables 5, 6 and 7.

Note: Care was taken to the best of our ability not to lose test substance, feed or water. Some loss was inevitable during procedures due to spillage.

The animals were examined for signs of toxicity twice daily throughout the 14-Day observation period. On Day 14 the animals were observed only once. The observations included the following: circulatory, respiratory, autonomic and central nervous systems; somatomotor activity; behaviour patterns; onset of tremors, convulsions, salivation, lethargy, diarrhea; skin and fur; and eyes and mucous membranes.

Fecal samples for both the test and control animals were collected on Days 1,3, 7, 10 and 14. They were stored frozen for a possible future analysis.

Body weights of the rats (recorded to the nearest tenth of a gram) were recorded daily. Initial and final weights as well as the change in total body weight are recorded in Tables 3 and 4.

At the end of the study, on Day 14, all rats were euthanized (by carbon dioxide) and gross necropsies were performed. Tissue samples for possible future histological examination were taken from each of the test animals. Tissues taken and preserved in 10% formalin were both kidneys, the fall gastrointestinal tract and sections of the liver. Necropsy observations are documented in Table 1. Additionally, the stomach contents were collected and stored frozen for possible future examination.

Results

All 20 test animals survived the 14 day observation period; however, all experienced weight loss. See Table 3 for specific weight losses. Observations were as follows:

Rat #1: Appeared unremarkable on Days 1 through 3. On Days 4 through 6, the animal had slight dehydration. On Days 7 through 14, the animal appeared unremarkable.

Rat #2: Appeared unremarkable on Days 1 through 3. On Day 4, the animal appeared dehydrated. On Day 5, the animal appeared unremarkable. On Day 6, the animal had slight dehydration. On Days 7 through 14, the animal appeared umremarkable.

Rat #3: Appeared unremarkable on Days 1 through 3. On Day 4, the animal had dull eyes and appeared dehydrated. On Day 5, the dehvdration remained. On Day 6, the animal was only slightly dehydrated. On Days 7 through 14, the animal appeared unremarkable.

Rat #4: Appeared unremarkable on Days I through 3. On Days 4 and 5, the animal was dehydrated. On Days 6 through 14, the animal appeared unremarkable.

Rat #5: Appeared unremarkable on Days 1 through 4. On Day 5, the animal exhibited slight dehydration On Days 6 through 14, the animal appeared unremarkable.

Rat #6: Appeared unremarkable on Days 1 through 3. On Day 4, the animal was slightly dehydrated. On Day 5, the animal had dull eyes and was dehydrated. On Day 6, the animal had slight dehydration. On Days 7 through 14, the animal appeared unremarkable.

Rat #8: Appeared unremarkable on Days 1 through 3. On Days 4 and 5, the animal was dehydrated. On Days 6 through 14, the animal appeared unremarkable.

Rat #9: Appeared unremarkable on Days ] through 3. On Days 4 and 5, the animal was dehydrated. On Days 6 through 14, the animal appeared unremarkable.

Rat #16: Appeared unremarkable throughout the 14 Day observation period.

Rat #17: Appeared unremarkable on Days 1 through 3. On Days 4 and 5, the animal appeared slightly dehydrated. On Days 6 through 14, the animal appeared unremarkable.

Rat #21: Appeared unremarkable on Days 1 through 3. On Day 4, the animal had dull eyes and appeared dehydrated. On Day 5, the animals had tremors, dull eyes and was dehydrated. On Days 6 ttu'ough 14, the animal appeared unremarkable.

Rat #24: Appeared unremarkable on Days 1 through 3. On Days 4 and 5, the animal appeared dehydrated. On Days 6 through 14, the animal appeared unremarkable.

Rat #26: Appeared unremarkable on Days 1 through 3. On Day 4, the animal had dull eyes and was dehydrated. On Day 5, the animal had rapid breathing, dull eyes and was dehydrated. On Day 6 the animal was slightly dehydrated. On Days 7 through 14, the animal appeared unremarkable.

Rat #27: Appeared unremarkable on Days 1 through 3. On Day 4, the animal appeared dehydrated. On Days 5 through 14, the animal appeared unremarkable.

Rat #29: Appeared unremarkable on Days I through 3. On Days 4 and 5, the animal was dehydrated. On Days 6 through 14, the animal appeared unremarkable.

Rat #30: Appeared unremarkable on Days 1 through 3. On Days 4 and 5, the animal had slight dehydration. On Days 6 through 14, the animal appeared unremarkable.

Rat #31: Appeared unremarkable on Days I through 3. On Days 4 and 5, the animal had slight dehydration. On Days 6 through 14, the animal appeared unremarkable.

Rat #32: Appeared umremarkable on Days I through 3. On Day 4, the animal had tremors, dull eyes, and was dehydrated. On Day 5, the animal had dull eyes and dehydration. On Days 6 through 14, the animal appeared unremarkable.

Rat #33: Appeared unremarkable on Days 1 through 4. On Day 5, the animal had slight dehydration. On Days 6through 14, the animals appeared unremarkable.

Rat #35: Appeared unremarkable on Days 1 through 3. On Days 4 and 5, the animal had slight dehydration. On Days 6 through 14, the animal appeared unremarkable.

All 10 of the control animals survived, gained weight, and appeared unremarkable during the 14 day observation period. See Table 4 for specific weight gains.

On Day 14, all 20 test rats were euthanized and necropsied. Three of the test animals had fluid- filled small intestines, one of which had a I×I cm2 pale yellow area on the stomach lining. All other test animals appeared unremarkable. Ten control animals were euthanized and necropsied. All control animals appeared normal. Statistical evaluation of the study data was not deemed necessary.

TABLE 3
Change in Rat Body Weights for Test Animals

| Animal | | Initial | Final | Change in |
|---|---|---|---|---|
| 1D No. | Sex | Day 0 (g) | Day 14 (g) | body wt |
| I | M | 153.6 | 136.5 | −17.1 |
| 2 | M | 160.6 | 125.4 | −35.2 |
| 3 | M | 156.5 | 131.4 | −25.1 |
| 4 | M | 169.6 | 151.8 | −I7.8 |
| 5 | M | 161.0 | 139.0 | −22.0 |
| 6 | M | 154.5 | 132.1 | −22.4 |
| 8 | M | 153.0 | 123.8 | −29.2 |
| 9 | M | 163.6 | 135.3 | −28.3 |
| 16 | M | 165.0 | 137.2 | −27.8 |
| 17 | M | 163.6 | 151.4 | −12.2 |
| 21 | F | 141.7 | 122.3 | −19.4 |
| 24 | F | 138.2 | 125.4 | −12.8 |
| 26 | F | 133.4 | 129.I | −4.3 |
| 27 | F | 140.0 | 135.0 | −5.0 |
| 29 | F | 141.8 | 131.5 | −10.3 |
| 30 | F | 148.2 | 131.8 | −16.4 |
| 31 | F | 134.0 | 123.7 | −10.3 |
| 32 | F | 145.1 | 131.5 | −13.6 |
| 33 | F | 130.7 | 124.1 | −6.6 |
| 35 | F | 136.9 | 124.1 | −12.8 |

TABLE 4
Change in Rat Body Weights for Control Animals Mal Final

| Animal | | Initial | Final | change in |
|---|---|---|---|---|
| lD No. | Sex | Day 0 (g) | Day 14 (g) | body wt |
| 13 | M | 158.2 | 245.4 | +87.2 |
| 14 | M | 155.5 | 256.1 | +100.6 |
| 15 | M | 161.5 | 263.1 | +100.6 |
| 18 | M | 155.5 | 270.1 | +114.6 |
| 19 | M | 160.0 | 263.7 | +103.7 |
| 23 | F | 145.2 | 187.6 | +42.4 |
| 28 | F | 139.5 | 179.5 | +40.0 |
| 34 | F | 134.0 | 170.0 | +36.0 |
| 36 | F | I39.2 | 186.2 | +47.0 |
| 37 | F | 134.3 | 183.6 | +49.3 |

TABLE 5
Average Food and Water Consumption for Test Animals

| | | Average wt of | Average wt of | Average vol of | Average vol of |
|---|---|---|---|---|---|
| Animal | | Food Consumed | Food Consumed | Water Consumed | Water Consumed |
| ID# | Sex | Prior to Test (g)¹ | While On Test (g)² | Prior to Test (ml) | While on Test (ml) |
| 1 | M | 16.0 | 29.3 | 21.8 | 39.8 |
| 2 | M | 17.1 | 26.1 | 27.1 | 37.1 |
| 3 | M | 16.9 | 26.0 | 22.8 | 36.2 |
| 4 | M | 18.1 | 25.9 | 23.8 | 35.9 |
| 5 | M | 17.4 | 27.4 | 24.2 | 38.0 |
| 6 | M | 16.5 | 27.4 | 22.2 | 37.5 |
| 8 | M | 14.3 | 27.8 | 19.1 | 37.0 |
| 9 | M | 16.6 | 25.9 | 21.5 | 33.7 |
| 16 | M | 18.2 | 27.8 | 21.6 | 35.3 |
| 17 | M | 16.2 | 27.8 | 22.9 | 37.1 |
| 21 | F | 14.1 | 27.3 | 22.3 | 39.6 |
| 24 | F | 14.7 | 25.8 | 20.8 | 35.53 |
| 26 | F | 15.0 | 27.2 | 19.3 | 40.3 |
| 27 | F | 15.7 | 26.7 | 21.9 | 39.3 |
| 29 | F | 14.4 | 29.0 | 18.7 | 39.4 |
| 30 | F | 16.8 | 25.6 | 25.9 | 35.7 |
| 31 | F | 16.3 | 29.3 | 19.3 | 37.6 |
| 32 | F | 16.1 | 30.1 | 27.64 | 39.9 |
| 33 | F | 14.9 | 30.2 | 21.8 | 41.6 |
| 35 | F | 14.3 | 24.7 | 22.0 | 34.0 |

¹Rats received certified Purina Rodent Chow
²Rats received the test substance, Eradirat, Lot #020901
³Calculation based on 13 days due to leaky water bottle
⁴Calculation based on 6 days due to leaky water bottle

TABLE 6
Average Food and Water Consumption for Control Animals

| | | Average wt of | Average wt of | Average wt of | Average wt of |
|---|---|---|---|---|---|
| Animal | | Food Consumed | Food Consumed | Water Consumed | Water Consumed |
| ID# | Sex | Prior to Test (g)* | While On Test (g)* | Prior to Test (ml) | While on Test (ml) |
| 13 | M | 14.4 | 21.6 | 23.0 | 29.3 |
| 14 | M | 15.6 | 24.7 | 21.9 | 28.3 |
| 15 | M | 18.3 | 25.4 | 25.2 | 30.4 |
| 18 | M | 16.2 | 25.3 | 21.0 | 29.1 |
| 19 | M | 15.0 | 26.8 | 21.4 | 26.6 |
| 23 | F | 15.4 | 17.7 | 22.8 | 22.2 |
| 28 | F | 14.1 | 17.1 | 18.0 | 9.4 |
| 34 | F | 16.8 | 16.1 | 24.5 | 26.0 |
| 36 | F | 13.5 | 16.1 | 16.4 | 21.6 |
| 37 | F | 16.5 | 17.7 | 21.4 | 23.3 |

*Rats received certified Purina Rodent Chow

TABLE 7
Food and Water Consumption Averages for All Animals

| | | | Average of All | |
|---|---|---|---|---|
| | Average of All Feed | Average of All Feed | Water Consumption | Average of All |
| | Consumption Prior | Consumption | Prior to Test | Water Consumption |
| | to Test Initiation (g) | During Test (g) | Initiation (ml) | During Test (ml) |
| Test Ma1es | 16.7 | 22.1 | 22.7 | 36.8 |
| Test Females | 15.2 | 27.6 | 22.0 | 38.3 |
| Control Males | 15.9 | 24.8 | 22.5 | 28.7 |
| Control Females | 15.3 | 16.9 | 20.6 | 22.5 |

### Summary

Eradirat, lot #020901, was administered to a group ot 20 white rats (10 males and 10 females) to evaluate its toxic characteristics in accordance with federal requirements as listed in 40 CFR 158, Subdivision F, Series 81-1. The animals were observed for 14 days. Any and all behavioral/clinical abnormalities, including mortalities, were recorded. On Day 14, all 20 rats were necropsied for gross pathology. At the time of necropsy, seventeen of the test rats appeared unremarkable, while three had fluid filled small intestines, one of which had a yellowish patch on the stomach lining.

Conclusion

The above study shows that ERADIRAT interferes with the digestion of laboratory rats and leads to dehydration and weight loss but not death under laboratory conditions.

In other laboratory studies, (on wild-caught Rattus norvegius rats) impaction of the caecum and intestine was noted on post mortem analysis following ingestion of a similar pelletised rodenticide in accordance with the invention. Caecal and intestinal compaction was noted in other laboratory studies following ingestion of a similar pelletised rodenticide in accordance with the invention.

1. A method of screening water-retentive candidate materials for potential rodenticidal activity in the field, the method comprising providing water-retentive materials as candidate materials, feeding rodents with the candidate materials ad libitum under laboratory conditions, measuring weight loss in the rodents during an initial phase of the testing, and selecting those candidate materials which lead to a mean weight loss of at least 15% of initial body weight.

2. A method according to claim 1 wherein those candidate materials which lead to a mean weight loss of at least 20% of initial body weight are selected.

3. A method according to claim 1 wherein those candidate materials which lead to a mean weight loss of at least 25% of initial body weight are selected.

4. A method according to claim 1 wherein those candidate materials which lead to a mean weight loss of at least 30% of initial body weight are selected.

5. A method according to any preceding claim wherein the rodents are laboratory rats or laboratory mice.

6. A method according to claim 6 wherein the rodents are rattus norvegicus.

7. A method of screening water-retentive materials for rodenticidal activity, wherein a water-retentive material is fed to rodents and the rodents are tested to determine whether or to what extent the water-retentive material has disrupted water transport through the wall of the gut.

8. A method according to claim 7 wherein the rodents are tested to determine whether or to what extent the water-retentive material has disrupted ion transport through the wall of the gut.

9. A method according to claim 7 or claim 8 wherein the effect of ingesting the water-retentive material on the size or condition of the thymus gland is tested.

10. A method according to any preceding claim wherein the rodents are examined for one or more of: bloating of the gut, impaction or compaction of the caecum and impaction or compaction of the intestine.

11. A method according to any preceding claim wherein said water-retentive material is of natural origin.

12. A method according to any preceding claim wherein said water-retentive material is cellulosic material.

13. A method according to claim 12 wherein the water-retentive material is derived from corn-cobs.

14. A method according to any preceding claim wherein the rodents are examined post mortem.

15. A method according to any preceding claim wherein the water-retentive material is substantially non-toxic to humans.

16. A rodenticide comprising a water-retentive material as the active ingredient and a rodent attractant.

17. A rodenticide according to claim 16 wherein the water-retentive material is cellulosic material.

18. A rodenticide according to claim 17 wherein the water-retentive material comprises alpha-cellulose.

19. A rodenticide according to claim 17 or claim 18 wherein the cellulosic material comprises purified cellulose derived from the core of the cob of the DK 446 maize hybrid or from the core of the cob of an agonist of the DK 446 hybrid.

20. A rodenticide comprising cellulosic water-retentive material as the active ingredient and a rodent attractant, the cellulosic water-retentive material being substantially free of corn-cob material.

21. A rodenticide comprising cellulosic water-retentive material as the active ingredient and a rodent attractant, the cellulosic water-retentive material being substantially free of material derived from the core of the cob of the DK 446 maize hybrid or from the core of the cob of an agonist of the DK 446 hybrid.

22. A rodenticide according to any of claims 15 to 21, further comprising a binder.

23. A rodenticide according to any of claims 15 to 22 which is in pellet form.

24. A rodenticide according to any of claims 15 to 23 wherein the bait attractant comprises sweet material.

25. A rodenticide according to claim 24 wherein the bait attractant comprises molasses.

26. A rodenticide according to any of claims 15 to 25 which in use disrupts ion transport through the wall of the gut of the rodent.

27. A moisture-proof container of rodenticide as claimed in any of claims 15 to 26.

28. A method of making a rodenticide comprising the steps of combining a water-retentive material with a rodent attractant, the water-retentive material being the active ingredient of the rodenticide.

29. A method according to claim 28 wherein the water-retentive material is cellulosic material.

30. A method according to claim 29 wherein the water-retentive material comprises alpha-cellulose.

31. A method according to claim 28 wherein the cellulosic material comprises purified cellulose derived from the core of the cob of the DK 446 maize hybrid or from the core of the cob of an agonist of the DK 446 hybrid.

32. A method according to any of claims 27 to 31 wherein at least the water-retentive material is dried under conditions of elevated temperature and/or pressure.

33. A method of making a rodenticide, the method comprising the step of combining cellulosic water-retentive material with a rodent attractant, the cellulosic water-retentive material being substantially free of corn-cob material and being the active ingredient of the rodenticide.

34. A method as claimed in claim 32, wherein the the cellulosic water-retentive material is substantially free of material derived from the core of the cob of the DK 446 maize hybrid or from the core of the cob of an agonist of the DK 446 hybrid.

35. A method according to any of claims 27 to 34 wherein the rodenticide is pelletised.

36. A method of screening water-retentive candidate materials for rodenticidal activity, substantially as described hereinabove with reference to Example 1 or Example 2.

37. A method of alleviating rodent infestation comprising depositing in an area of rodent infestation a rodenticide as claimed in claim 16, the rodenticide being non-toxic to humans and disrupting the digestion of the rodents on being ingested.
